(19) United States
(12) Patent Application Publication
(10) Pub. No.: US 2024/0226329 A1
(43) Pub. Date: Jul. 11, 2024

(54) BACTERIAL TARGETING VECTOR CARRYING CYTOKINE OR POLYNUCLEOTIDE THEREOF AND USE THEREOF IN TUMOR TREATMENT

(71) Applicant: SHENZHEN INSTITUTES OF ADVANCED TECHNOLOGY CHINESE ACADEMY OF SCIENCES, Guangdong (CN)

(72) Inventors: Chenli Liu, Shenzhen (CN); Yuxuan Dong, Shenzhen (CN); Xuan Guo, Shenzhen (CN); Qian Chen, Shenzhen (CN); Yong Hu, Shenzhen (CN); Liangxia Ai, Shenzhen (CN); Hao Zhang, Shenzhen (CN)

(73) Assignee: SHENZHEN INSTITUTES OF ADVANCED TECHNOLOGY CHINESE ACADEMY OF SCIENCES, Guangdong (CN)

(21) Appl. No.: 18/022,051

(22) PCT Filed: Aug. 20, 2020
(86) PCT No.: PCT/CN2020/110159

§ 371 Date: Oct. 12, 2023

(30) Foreign Application Priority Data
Aug. 18, 2020 (CN) 202010830264.5

(51) Int. Cl. A61K 48/00; A61K 35/74; A61K 38/20; A61K 45/06

(52) U.S. Cl. CPC A61K 48/0058; A61K 35/74; A61K 38/204; A61K 38/2066; A61K 45/06

(57) ABSTRACT

A bacterial targeting vector and use thereof in tumor treatment, in particular, a bacterial targeting vector carrying a cytokine such as interleukin 6 and interleukin 10 or carrying a polynucleotide such as mRNA of the cytokine and use thereof in tumor treatment. An attenuated chassis bacterium capable of targeting solid tumors can be used as a delivery carrier of a tumor therapy molecule, and achieve expression and controllable release of a human/murine cytokine, thereby achieving anti-tumor effects.

### CROSS-REFERENCE OF RELEVANT REFERENCES

The application claims the priority to Chinese Patent Application NO. 202010830264.5 filed on Aug. 18, 2020, with the titled “Bacterial targeting carrier carrying a cytokine or the polynucleotide thereof and its use in tumor treatment”, which is incorporated herein by reference in its entirety.

### FIELD OF THE INVENTION

The invention relates to the field of tumor targeting therapy, in particular, to a bacterial targeting carrier carrying a cytokine, especially interleukin-6 or interleukin-10, or a polynucleotide encoding the cytokine such as mRNA and the use thereof in tumor treatment.

### BACKGROUND OF THE INVENTION

Currently, all the conventional tumor therapies such as operative therapy, radiotherapy and chemotherapy etc., can no longer meet the clinical needs of patients due to the deficiencies as revealed, such as damage to normal tissues and severe toxic adverse effects and the like. Emerging therapies such as CAR-T, checkpoint inhibitor bring new hope to tumor treatment. However, the immunotherapy for tumor is somewhat limited, and has less therapeutic effect on solid tumors, which limits the further development and application of immunotherapy for tumor. Therefore, the key for achieving breakthroughs in immunotherapy is how to expand the range of choice for immunotherapy.

The studying history of bacteriotherapy for tumor can be traced back to 150 years ago. The bacteriotherapy for tumor relies on characteristics of bacteria, such as wide variety, motility, tumor tropism, invasion ability, cytotoxicity and the like, to achieve tumor intervention. Living bacteria have a unique capability to actively target tumors and specifically colonize in tumors, and can target most of tumors including metastatic tumor. Living bacteria may better infiltrate tumor tissues relying on flagella. Although traditional anticancer medicaments have strong therapeutic-toxic efficacy, they pervade throughout the body with blood circulation system, resulting in the death of a great number of normal cells by indiscriminately attack. Complex tumor vasculature hinders the deep delivery of traditional anticancer medicaments, making it difficult to play a role in tumor killing. Therefore, the combination of bacteriotherapy and traditional therapy will complement the shortcomings of each other.

The development and maturation of gene engineering technology and concept of synthetic biology provide a more effective and safer therapeutic strategy for combining bacteriotherapy and immunotherapy for tumor. Genetically engineered bacteria can express a molecule with antitumor activity, such as a cytokine and the like, and can achieve targeted expressions of the molecule with antitumor activity in tumor regions with the advantages such as natural tumor targeting of bacteria and the like. The bacteria can exhibit excellent properties such as specifically targeting tumors, controlled medicament release, significant tumor intervention and stronger safety upon the optimization and reconstruction of the chassis cell by designing and synthesizing functional (targeting, regulating and the like) modules under technical concept of synthetic biology.

Currently, the examples for the combination of bacteriotherapy and immunotherapy have been reported in several articles. Attenuated bacteria were engineered to deliver antitumor agents such as cytokines and the like, thereby further enhancing the antitumor effect. Xiao-qin Ha et al. (“Inhibitory Effects of the Attenuated Salmonella typhimurium Containing the IL-2 Gene on Hepatic Tumors in Mice”, Journal of Biomedicine and Biotechnology, Volume 2012, Article ID 946139) demonstrated that attenuated Salmonella expressing IL-2 had better potential in inhibiting the proliferation of hepatoma cell as compared with control Salmonella. Similar to the work about IL-2, M Loeffler et al. 2008 (“IL-18-producing Salmonella inhibit tumor growth”, Cancer Gene Therapy (2008) 15, 787-794) reported attenuated Salmonella typhimurium expressing IL-18. In situ expressions of cytokines such as LIGHT. IL-18, CCL21 and the like in solid tumors activate immunosuppressive environment in tumors by inducing the proliferation of T cell, dendritic cell, natural killer cell and the like in tumor region, playing a role in the intervention of solid tumors such as liver cancer and the inhibiton of metastatic tumors. In addition, researchers from Chonnam National University used engineered Salmonella to develop a constitutive therapeutic system. Attenuated ΔppGpp Salmonella typhimurium was genetically engineered, making it secrete flagellin FlaB from Vibrio vulnificus. The synergetic activation of TLR5 and TLR4 signal pathways increased the secretion of tumor suppressor, and significantly enhanced the killing capacity of Salmonella against colorectal cancer cells.

Chinese patent application CN102526760A discloses a vaccine and a pharmaceutical composition of recombinant attenuated Salmonella for treating solid tumors, wherein the recombinant attenuated Salmonella vaccine comprises a eukaryotic expression plasmid carrying dual genes interleukin 2 and hepatocyte growth factor antagonist NK4, therefore, it, in essence, released the plasmid to the human body to achieve eukaryotic expression.

PCT patent application WO2015032165A1 discloses a bacterial delivery and expression system targeting tumors, which was used for prokaryotic-eukaryotic delivery and expressing therapeutic molecules in mammal cells. The delivered and expressed therapeutic agent was pore-forming listeriolysin, and the purpose of delivering and releasing therapeutic agents with prokaryotic vector was not achieved.

Christoph Pöhlmann et al. (“Periplasmic Delivery of Biologically Active Human Interleukin-10 in Escherichia coli via a Sec-Dependent Signal Peptide”, J Mol Microbiol Biotechnol 2012; 22:1-9) used E.coli to secrete and express bioactive human-derived cytokine interleukin 10, which was used for treating inflammatory bowel disease. The eukaryotic signal sequence of human interleukin 10 was replaced with the signal peptide from ompF gene cloned from E. coli K-12 MG1655, therefore, achieving the fusion of prokaryotic signal peptide from ompF gene and human interleukin 10, which was cloned into BAC pSG1107 plasmid vector with the expression thereof initiated by T7 promoter, achieving the transmembrane delivery of heterologous proteins, directing the extracellular secretion. Upon the expression in E. coli BL21(DE3), the expression in culture supernatant and intracellular expression were 5.8±1.7 and 355.8±86.3 ng/ml, respectively.

Currently, there is not a prokaryotic carrier optimized for the precise delivery and release of cytokines to targeted tumors, and the therapeutic use of the carrier.

### SUMMARY OF THE INVENTION

To solve the problem in prior art, the present invention intends to provide a prokaryotic delivery carrier targeting a tumor and use thereof in tumor treatment.

In the first aspect, the invention provides a prokaryotic delivery carrier targeting a tumor, which is a chassis bacterium targeting the anaerobic area of a solid tumor, and comprises a polynucleotide for expressing a cytokine, wherein,
the cytokine is selected from the group consisting of interleukin 6, interleukin 10, interleukin8, interleukin 18, interleukin 33 and TNF-α, and
the polynucleotide is DNA or mRNA.

In the first aspect of the invention, the chassis bacterium is selected from the group consisting of attenuated Salmonella typhimurium (Salmonella enterica serovars typhimurium), attenuated Salmonella typhi (Salmonella enterica serovars typhi), Escherichia coli (E.coli) and lactic acid bacteria.

In the first aspect of the invention, the chassis bacterium is attenuated Salmonella typhimurium.

In the first aspect of the invention, the polynucleotide is DNA, and comprises signal peptides pelB and OmpF fused to the DNA expressing the cytokine.

In the first aspect of the invention, the polynucleotide is mRNA, and the mRNA further comprises an m7G capping structure at the 5′ end and polyA tail structure at the 3′ end.

In the first aspect of the invention, the cytokines are selected from the group consisting of interleukin 6 and interleukin 10.

In the first aspect of the invention, the prokaryotic delivery carrier comprises a promoter which is sensitive to tumor environment. The may also comprises other promoters which are constitutive promoter and inducible promoter, the inducible promoter is, e.g., an IPTG inducible promoter or an aTc inducible promoter.

In the first aspect of the invention, the tumor is a solid tumor found in the following sites: cervix uteri, breast, prostate, gastrointestinal tract (e.g., esophagus, oropharynx, stomach, small intestine or large intestine, colon, rectum), bladder, bone, skin, head or neck, liver, gall bladder, lung, pancreas, salivary gland, adrenal gland, thyroid, cerebral (e.g., glioma) ganglion, the tumor is preferably colon cancer, breast cancer, melanoma, lung cancer, glioblastoma.

In the first aspect of the invention, the carrier is administered by intramuscular injection, intravenous injection, subcutaneous injection, intraperitoneal injection, intracerebral administration, intraoral administration, intranasal administration or oral administration.

In the second aspect, the invention provides use of the prokaryotic delivery carrier as described above in tumor treatment, wherein, the prokaryotic delivery carrier is combined with a therapeutic agent selected from the group consisting of chemical medicaments, e.g., alkylating agent (such as thiotepa and cyclophosphamide), alkane sulfonate, Az-cycloalkene medicaments, ethylamine and methylamine, nitrogen mustards, antibiotics, purine analogue, pyrimidine analogue, androsterone, antiadrenergic medicament, topoisomerase inhibitor, capecitabine; chemotherapeutics: anti-hormone agents regulating or inhibiting the effect of hormones on tumor; immuno/targeting medicaments: Gefitinib, Iressa, Lorlatinib, Erlotinib, Bevacizumab.

In the second aspect of the invention, the combination is simultaneous administration, or sequential administration, or separate administration.

The attenuated chassis bacterium targeting a solid tumor of the invention can be used as a delivery carrier for a therapeutic molecule for tumor, and achieve the expression and controlled release of a human/murine cytokine, thereby achieving anti-tumor effects.

### BRIEF DESCRIPTION OF THE DRAWINGS

FIG. 1 is the expressions of IL-10 protein observed at the indicated time after transfecting 293T cells with IL10 cmRNA prepared in Example 1 at different concentrations.

FIG. 2 is the maps of the original plasmid used in Example 2 and constructed plasmid. FIG. 2 (left) is the original plasmid, pSC101-GFP-Kana; Example 2 (right) is the plasmid with signal peptide inserted, pSC101-pelB-GFP-Kana.

FIG. 3 is the expression at RNA level by plasmid pSC101-pelB-GFP-Kana as constructed in Example 2.

FIG. 4 is the map of the plasmid constructed by Example 3. FIG. 4 (left) is plasmid pSC101-pelB-IL-6-Kana; FIG. 4 (right) is plasmid pSC101-pelB-IL-10-Kana.

FIG. 5 is the construction of the plasmid of the invention with interleukin 10 carrying the eukaryotic signal peptide thereof (pET30a-IL10 carrying eukaryotic signal peptide) and the result of sequencing.

FIG. 6 is the construction of the plasmid of the invention with interleukin 10 carrying the E.coli signal peptide thereof (pET30a-IL10 carrying E.coli ompF signal peptide) and the result of sequencing.

FIG. 7 is the construction of the plasmid of the invention with interleukin 10 carrying the Salmonella signal peptide thereof (pET30a-IL10 carrying Salmonella ompF signal peptide) and the result of sequencing.

FIG. 8 is the construction of the plasmid of the invention with interleukin 10 carrying the pelB signal peptide thereof (pET22b-IL 10 carrying pelB signal peptide) and the result of sequencing

FIG. 9-11 are the identification of bacterial suspension by PCR after transfecting the cells with 4 plasmids prepared in Example 4, respectively.

FIG. 12 shows the expression levels of IL-10 carrying different signal peptides in BL21(DE3). The expression level of IL-10 in the supernatant from the lysate of BL21(DE3) carrying different signal peptides (left) and the expression level in LB medium supernatant (right). BL21(DE3)-pET22b(+)-pelB-IL-10 (indicated as pelB in the figure), BL21(DE3)-pET30a-ompF-IL-10 (indicated by ompF in the figure), BL21(DE3)-pET30a-IL-10 (indicated by eukaryon in the figure).

### DETAILED DESCRIPTION OF THE INVENTION

Although various modifications can be made to the invention and there may be a variety of forms for the invention, the embodiments will be described and interpreted in details as follows. However, it will be understood that these do not intend to limit the invention to the particular disclosure, and the invention includes all modifications, equivalents or alternatives while not departing from the spirit and scope of the invention.

The prokaryotic delivery carrier targeting a tumor of the invention and use thereof in tumor treatment will be interpreted according to the specific embodiments in more details hereinafter.

In one or more embodiments of the invention, the prokaryotic delivery carrier targeting a tumor of the invention is a chassis bacterium targeting the anaerobic area of a solid tumor, and comprises a polynucleotide for expressing a cytokine, wherein the cytokine is selected from the group consisting of interleukin 6, interleukin 10, interleukin 8, interleukin 18, interleukin 33 and TNF-a, preferably interleukin 6 or interleukin 10. The polynucleotide is DNA or mRNA.

In one or more embodiments of the invention, the invention is applied to use of the prokaryotic delivery carrier in tumor treatment, wherein the prokaryotic delivery carrier is combined with a therapeutic agent selected from the group consisting of chemical medicaments, e.g., alkylating agent (such as thiotepa and cyclophosphamide), alkane sulfonate, Az-cycloalkene medicaments, ethylamine and methylamine, nitrogen mustards, antibiotics, purine analogue, pyrimidine analogue, androsterone, antiadrenergic medicament, topoisomerase inhibitor, capecitabine; chemotherapeutics: anti-hormone agents regulating or inhibiting the effect of hormones on tumor; immuno/targeting medicaments: Gefitinib, Iressa, Lorlatinib, Erlotinib, Bevacizumab. The combination is simultaneous administration, or sequential administration, or separate administration.

In one or more embodiments of the invention, the invention provides a method for treating a tumor with prokaryotic delivery carrier targeting a tumor of the invention, especially a chassis bacterium the anaerobic area of a solid tumor, and comprises a polynucleotide for expressing a cytokine, wherein the cytokine is selected from the group consisting of interleukin 6, interleukin 10, interleukin 8, interleukin 18, interleukin 33 and TNF-α, preferably interleukin 6 or interleukin 10. The polynucleotide is DNA or mRNA

In one or more embodiments of the invention, the prokaryotic delivery carrier may be combined with a therapeutic agent selected from the group consisting of chemical medicaments, e.g., alkylating agent (such as thiotepa and cyclophosphamide), alkane sulfonate, Az-cycloalkene medicaments, ethylamine and methylamine, nitrogen mustards, antibiotics, purine analogue, pyrimidine analogue, androsterone, antiadrenergic medicament, topoisomerase inhibitor, capecitabine; chemotherapeutics: anti-hormone agents regulating or inhibiting the effect of hormones on tumor; immuno/targeting medicaments: Gefitinib, Iressa, Lorlatinib, Erlotinib, Bevacizumab. The combination is simultaneous administration, or sequential administration, or separate administration.

In one or more embodiments of the invention, the invention provides use of prokaryotic delivery carrier of the invention in the preparation of a medicament for treating a tumor, wherein the prokaryotic delivery carrier is combined with a therapeutic agent selected from the group consisting of chemical medicaments, e.g., alkylating agent (such as thiotepa and cyclophosphamide), alkane sulfonate, Az-cycloalkene medicaments, ethylamine and methylamine, nitrogen mustards, antibiotics, purine analogue, pyrimidine analogue, androsterone, antiadrenergic medicament, topoisomerase inhibitor, capecitabine; chemotherapeutics: anti-hormone agents regulating or inhibiting the effect of hormones on tumor; immuno/targeting medicaments: Gefitinib, Iressa, Lorlatinib, Erlotinib, Bevacizumab. The combination is simultaneous administration, or sequential administration, or separate administration.

In one or more embodiments of the invention, the chassis bacterium is selected from the group consisting of attenuated Salmonella typhimurium (Salmonella enterica serovars typhimurium), attenuated Salmonella typhi (Salmonella enterica serovars typhi), Escherichia coli (E.coli) and lactic acid bacteria.

In one or more embodiments of the invention, the polynucleotide is DNA, and the carrier further comprises a pelB signal peptide fused to the DNA expressing the cytokine. The carrier of the invention further comprises a promoter which is sensitive to tumor environment.

In one or more embodiments of the invention, the polynucleotide is mRNA, the mRNA further comprises an m7G capping structure at the 5′ end and poly A tail structure at the 3′ end.

In one or more embodiments of the invention, the tumor is a solid tumor found in the following sites: cervix uteri, breast, prostate, gastrointestinal tract (e.g., esophagus, oropharynx, stomach, small intestine or large intestine, colon, rectum), bladder, bone, skin, head or neck, liver, gall bladder, lung, pancreas, salivary gland, adrenal gland, thyroid, cerebral (e.g., glioma) ganglion, the tumor is preferably colon cancer, breast cancer, melanoma, lung cancer, glioblastoma.

In one or more embodiments of the invention, the carrier or medicament is administered by intramuscular injection, intravenous injection, subcutaneous injection, intraperitoneal injection, intracerebral administration, intraoral administration, intranasal administration or oral administration.

The prokaryotic delivery carrier of the invention can be constructed and verified by the follows: (1) modifying the cytokine mRNA with chemical modification; (2) delivering the chemically modified cytokine mRNA with a bacterial carrier; (3) the construction of a plasmid vector system for heterologously expressing the cytokine; (4) identifying the expression of the cytokine by ELISA or Western Blot; (5) in vitro experiment to testing the function of the bacterium in secreting the cytokine; (6) testing the effect of interleukin 10 on treating a tumor with tumor-bearing mouse model.

The cytokine mRNA of the invention is present in the bacterium in the form of a plasmid, which is released for expression after the bacterium is delivered into a tumor, and is translated in the tumor. The chemical modification can significantly prolong the time of degradation, and avoid degradation.

One aspect of the invention relates to a method using a modified mRNA for interleukin 6 or interleukin 10 cytokine which is transcribed in vitro, comprising introducing it into a chassis microbe, thus entering solid tumor through blood vessel via the bacterium, and achieving secretion and translation of the mRNA in solid tumor.

In particular, e.g., the invention can achieve the modification and in vitro transcription of the cytokine mRNA by the construction of a system for delivering and expressing chemically modified cytokine mRNA with a bacterial carrier. To achieve an effective in vitro translation of mRNA, the invention incorporates an m7G capping structure at the 5′ end and a polyA tail structure at the 3′ end when in vitro transcribing and synthesizing mRNA. The capping process is performed by cotranscription with T7 RNA polymerase; and the poly(A) tail is added at the 3′ end of the mRNA molecule with poly(A) polymerase in the tailing process. Then, DNA template is digested with DNase. cmRNA is expressed from a plasmid, which is electro-transferred into a chassis bacterium, thus achieving the transmembrane secretion of cmRNA, and then, the production of IL-6/IL-10 protein by extracellular translation.

Another aspect of the invention relates to achieving the expression and extracellular secretion of a target protein with a signal peptide fused to a cytokine.

In particular, the fusion of the signal peptide to the cytokine and the expression are achieved by the construction of a plasmid vector system construction for the heterologous expression of murine cytokines IL-6 and IL-10 in a bacterium. Inclusion bodies are greatly formed in the heterologous expression of a cytokine. In order to avoid the emergence of this problem, the invention intends to express the target gene fused to a signal peptide. A signal peptide can direct the extracellular transport of a synthesized protein. The invention employs the signal peptide pelB derived from the pectate lyase of Erwinia carotovora for the study of the invention. In addition, the invention optionally employs a purification tag, 6×His tag, as the marker for the subsequent detection of the target gene. IL-6 and IL-10 in bacterial supernatant are detected by enzyme-linked immunosorbent assay (ELISA), thus verifying whether the extracellular secretion of IL-6 and IL-10 is achieved.

With respect to the system for the chemically modified cytokine mRNA, and the plasmid vector system for the heterologous expression of murine cytokines IL-6 and IL-10 in a bacterium as constructed above, the activity of the expressed cytokines IL-6 and IL-10 can be determined according to the following solid phase enzyme-linked immunospot technique and/or the test for the therapeutic effect of the cytokines IL-6 and IL-10 secreted by the bacteria on tumor-bearing mice.

The solid phase enzyme-linked immunospot technique (ELISPOT) is carried out to identify, in vitro, the function of cytokines IL-6 and IL-10, i.e., to confirm whether the cytokines IL-6 and IL-10 secreted by the bacteria have the T cell activating function. The invention employs CD4+T cell and CD8+T cell in culture in vitro, and the activation of T cell is detected by the solid phase enzyme-linked immunospot technique (ELISPOT). Meanwhile, control experiments are designed, in which, T cells, CD4+T cell and CD8+T cell cultured in vitro, are directly stimulated with cytokines IL-6 and IL-10.

The test for therapeutic effect of cytokines IL-6 and IL-10 secreted by bacteria on tumor-bearing mice. To demonstrate the inhibition of attenuated Salmonella expressing cytokines IL-6 and IL-10 on solid tumor, in the invention, macrophage-deficient mice was constructed to eliminate the interference of factors such as IL-10 secreted by macrophage, the tumor size in mice was observed, and the secretion of cytokines such as TNF-α, IFN-γ was detected upon the collection of tumor sera.

The invention can also employ the delivery system constructed based on pET-22b(+) and pET30a by modification.

The promoter in the invention can be a constitutive promoter J23101 and a IPTG or aTc inducible expression promoter.

The ribosome-binding site (RBS) in the invention can be B0034, and RBSs with different strength for expression, including but not limited to BBa J61100, BBa J61101, BBa J61139, etc., which are more than 40. These RBS can be used to characterize the expression intensity of target protein.

The chassis microorganism of the invention can be E. coli BL21(DE3), Nissle 1917 and Salmonella SL7207 (aroA−), etc.

The above various embodiments of the invention have the following benefits:
(1) the use of the cytokine interleukin 10 expressed in fusion to a signal peptide in tumor treatment exploits a new application;
(2) the employed chassis microorganism is essentially attenuated Salmonella, showing significant killing effect on solid tumor;
(3) the half-life of the cytokine is effectively increased, the duration is prolonged, and the therapeutic effect is improved in the method with chemically modified mRNA of cytokines interleukin 6 or interleukin 10;
(4) the expression of cytokine interleukin 6 or interleukin 10 by the bacterial carrier constructed in the invention is higher.

### EXAMPLES

The following non-limiting Examples are provided.

### Example 1: The Preparation and Intracellular Expression of IL-10 mRNA

IL-10 DNA, as the template, was constructed on the plasmid having a T7 promoter, which was linearized to serve as a further template for the synthesis of RNA by in vitro transcriptional reaction, the m7G capping structure at the 5′ end and poly A tail structure at the 3′ end were added in the synthetic reaction, and m5C, Pseudo-UTP and other chemically modified bases were simultaneously added to increase the stability and translation efficiency. The capping process was carried out by in vitro transcription through T7 RNA polymerase at 37° C. for 6 h, in which lug template DNA was added to 20 μl in vitro transcription system, and while adding 150 nmol ATP, m5C and Pseudo-UTP, ARCA at a ratio of 4:1 (ARCA:GTP=4:1, 150 nmol in total) was added. Next, the DNA template was digested by TURBO DNase at 37° C. for 30 min, followed by tailing reaction for 1 h. The reaction system was expanded by 5 times, 100 nmol ATP and 250 nmol MnCl₂ were exogenously added to the 100 μl reaction system, polyadenylate was synthesized at the 3′ end of RNA with Poly(A) polymerase under the reaction condition at 37° C. Finally, the synthesized mRNA was purified by LiCl precipitation.

To confirm whether IL-10 mRNA can normally translated in cells, the chemically modified cmRNA was transfected into 293T cells at transfection doses of 0.5 μg and 1.5 μg per well, respectively, the lysate and supernatant were extracted in 24 h, 36 h, 48 h, 60 h, respectively, with starting time point at 24 h after cell incubation. The expression of IL-10 protein was determined by ELISA, and the results were shown in FIG. 1 and Table 1. Table 1 showed the expression of IL-10 in the transfected 293T cells (pg/mL).

It can be seen from FIG. 1 and Table 1 that, compared with the control cells which were not transfected with the RNA, the expression of IL-10 protein in the lysate from cells transfected with RNA was significantly increased, suggesting that IL-10 cmRNA was normally translated in cells; and the expression of IL-10 protein in the supernatant was at a relatively high level, suggesting that the protein was normally translated and was secreted out of cells in large quantities.

TABLE 1

| | | 0.5- | | 1.5- | | control - |
|---|---|---|---|---|---|---|
| time(h) | 0.5 | supernatant | 1.5 | supernatant | control | supernatant |
| 24 | 2093.86 | 3408.14 | 3071.00 | 3245.29 | 85.29 | −39 |
| 36 | 1741.00 | | 2449.57 | | | |
| 45 | 2115.29 | 3585.29 | 2882.43 | 3553.86 | | |
| 60 | 2479.57 | | 2202.43 | | | |

### Example 2: Functional Identification of pelB Signal Peptide with GFP as a Target Gene

### (1) Construction of pSC101-pelB-GFP-kana Plasmid

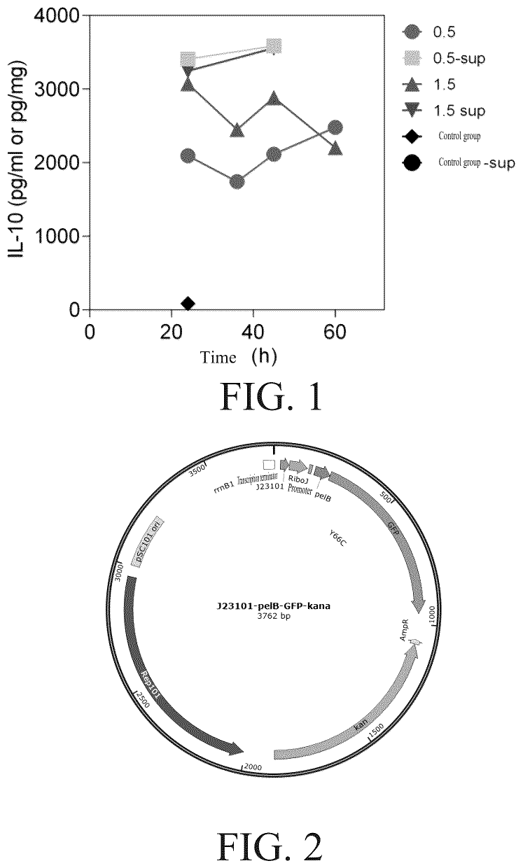

pSC101-GFP-Kana plasmid in iGEM Gene element library of International Genetically Engineered Machine competition (UT Austin iGEM 2019: Characterization of metabolic burden of the Anderson Series, see website http://parts.igem.org/wiki/index.php?title=Part:BBa_J23101) was selected as template, and the expression of the target gene GFP in the plasmid was initiated by J23101 constitutive promoter. By means of One Step-Cloning, forward and reverse primers were designed to amplify signal peptide pelB and the backbone fragment, and the pelB fragment was inserted to the plasmid by means of homologous recombination to obtain pSC101-pelB-GFP-kana plasmid as shown in FIG. 2 below. FIG. 2 (left) was the original plasmid template, pSC101-GFP-Kana; and FIG. 2 (right) was the plasmid with inserted signal peptide, pSC101-pelB-GFP-Kana.

### (2) Functional Identification of pelB Signal at RNA Level

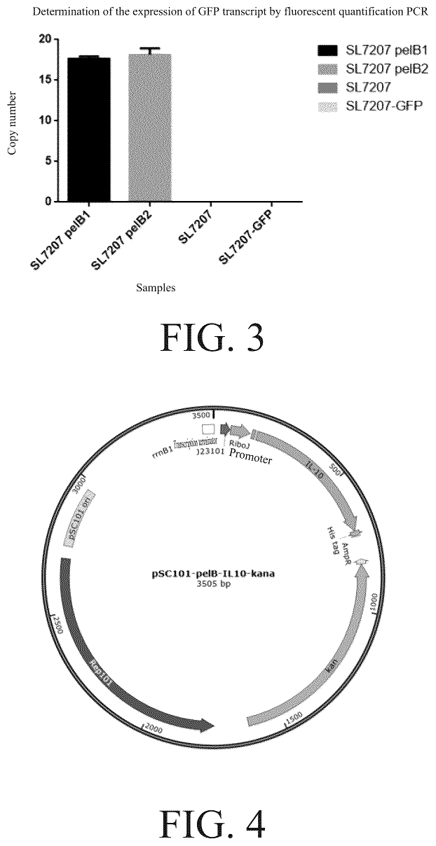

The plasmid constructed above was electro-transformed to attenuated Salmonella typhimurium (reference is made to attenuated Salmonella YB1, disclosed by Chang-Xian Li et al., Oncology Letters, Jan 2017, Volume 13 Issue 1, DOI: 10.3892/ol.2016.5453), and the GFP expression was detected at RNA level. The GFP expression at RNA level was analyzed by real-time PCR. The forward and reverse primers qP-pelB-F1/R1 were designed for real-time PCR with sequences of GGCAGCCGCTGGATTGTTAT/TGTTGCATCACCTTCACCCT and a Tm of 60° C.; the test was carried out with SL7207 (aroA−) and SL7207 transferred with the original template plasmid selected as controls, meanwhile, the sample prior to reverse transcription was selected as control to avoid errors caused by the genomic DNA which had not been completely removed. FIG. 3 showed GFP expression at RNA level. The result was shown in FIG. 3, when testing with Real-time PCR, rho was selected as the internal reference gene, the ratio of sample result data to rho internal reference gene was calculated, the result was as follows: in the bacterium electro-transformed with the plasmid PSC101-pelB-GFP-Kana, the GFP expression was high at mRNA level, the controls, both SL7207 and SL7207 transformed with the original template plasmid, did not show mRNA expression.

### Example 3: Construction of Plasmid Delivery System with IL-6 and IL-10 as Target Genes

The pSC101-pelB-GFP-Kana plasmid as described above was selected as template, and forward and reverse primers were reversely designed for amplifying the backbone, with the forward primer having the sequence of 5′-GTCGACACTTAATTAACGGCA-3′; and the reverse primer having the sequence of 5′-CTCGAGATTTCTCCTCTTTCG-3′. Murine IL-10 gene was amplified from Puc-IL-10-AMP plasmid with the forward primer have the sequence of 5′-GAAAGAGGAGAAATCTCGAGATGA-3′; and the reverse primer having the sequence 5′-GCCGTTAATTAAGTGTCGACAC-3′. By means of One Step-Cloning, the target fragment was inserted into the backbone to obtain pSC101-pelB-IL-10-kana plasmid.

The enzyme PrimerSTAR MAX Premix (2×) from Takara, and dNTP at a concentration of 0.4 mM (2×) were used for the high-fidelity amplification by PCR, and the PCR reaction condition was as follows:

| | system | | | program |
|---|---|---|---|---|
| | template: plasmid pET-22b | 2 μl | | 1: 98° C. 5 min |
| | enzyme: 2 × pro Taq (AG) | 10 μl | | 2: 98° C. 10 s |
| {open oversize brace} | primer 1 | 0.4 μl | | 3: 55° C. 30 s |
| | primer 2 | 0.4 μl | {open oversize brace} | 4: 72° C. 6 min |
| | ddH2O: RNase-free ddH₂O | 7.2 μl | | 30 cycles of steps 2-4 |
| | | | | 5: 72° C. 5 min |
| | | | | 6: 12° C. ∞ |

pSC101-pelB-IL-6-kana was constructed by the same way, as shown in FIG. 4.

### Example 4: Construction, Transformation and Expression of pET30a-IL10 Carrying ompF Signal Peptide and pET22b-IL10 Carrying pelB Signal Peptide

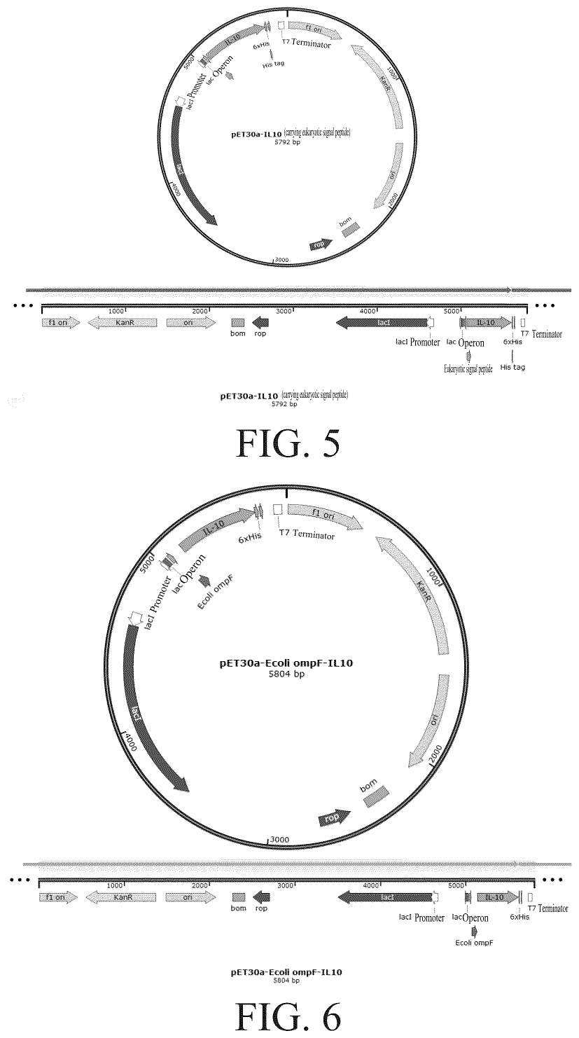

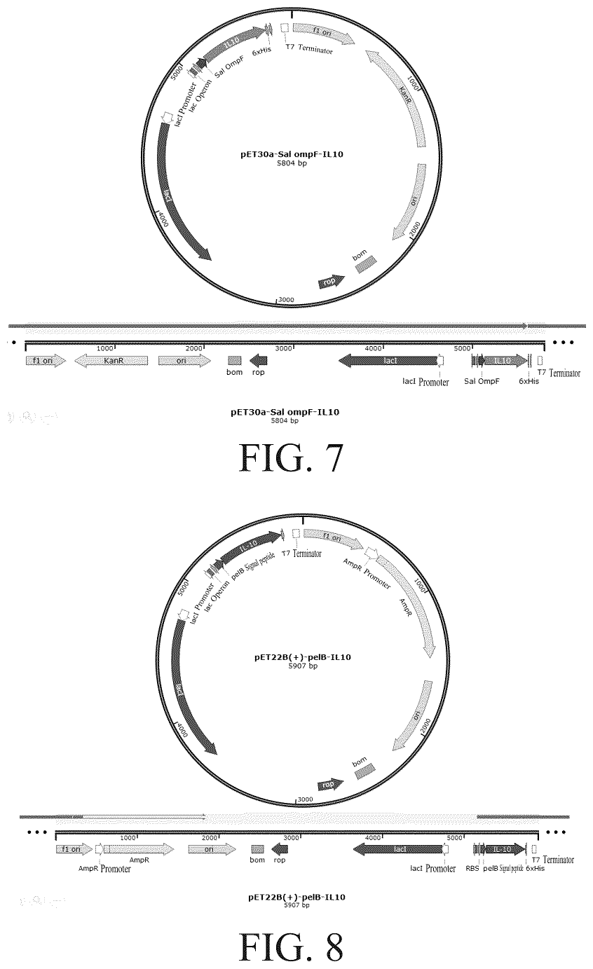

Construction: Upon an analysis of the sequence of IL-10 protein, it was found that the first 22 amino acids of IL-10 were an eukaryotic signal peptide. In order to facilitate successful expression and transmembrane delivery of IL-10 in prokaryotic carrier, the applicant replaced the original eukaryotic signal peptide with prokaryotic signal peptides pelB and ompF, and used inducible plasmid expression vector to increase the soluble expression of the target protein. The plasmids were successfully constructed: pET30a-IL10 carrying ompF signal peptide and pET22b-IL10 carrying pelB signal peptide. pET30a and pET22b were common IPTG inducible expression vectors. The constructed plasmids were shown in FIG. 5-8.

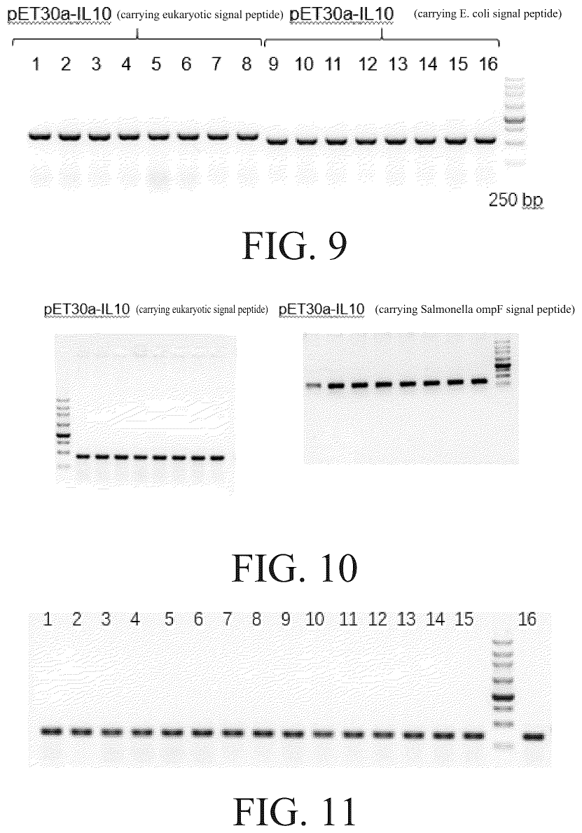

Transformation: The above-described plasmids were electro-transformed into BL21(DE3) and attenuated Salmonella ZG1, respectively. The pET30a-IL10 plasmid carrying eukaryotic signal peptide were constructed in BL21(DE3) and ZG1, respectively, to achieve strains capable of normally expressing IL-10. In addition, pET30a-IL10 carrying E.coli ompF signal peptide and pET30a-IL10 carrying Salmonella ompF signal peptide were constructed in BL21(DE3) and ZG1 to achieve the expression and cytoplasmic secretion of IL-10; and the pET22b-IL 10 plasmid carrying pelB signal peptide was constructed in BL21(DE3) and ZG1 to achieve the expression and cytoplasmic secretion of IL-10. The identification by bacterial PCR was shown in FIGS. 9-11.

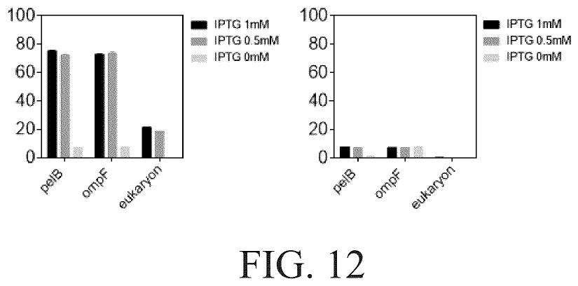

E.coli BL21(DE3) carrying pET30a-ompF-IL10 and pET22b(+)-pelB-IL10 plasmids as well as E.coli BL21(DE3) carrying pET30a-IL10 having eukaryotic signal peptide used as control were cultured in LB media in an expanded scale with a culture volume of 4 ml, respectively. When bacteria was cultured to late logarithmic phase (OD value of about 0.6), IPTG was added at a final concentration of 1, 0.5, or 0 mM, followed by the expression under induction at 16° C. overnight (24 h). The bacteria and LB medium were collected. The bacteria were lysed completely with protein lysis buffer, followed by centrifugation for 30 min at 14000 g. The supernatant of the lysate and supernatant of LB medium were collected, respectively. The expression of interleukin 10 was quantitatively analyzed by immunofluorescence adsorption ELISA. The result was shown in FIG. 12. Conclusion: It was demonstrated that the expression level of soluble IL-10 protein of 70-80 ng/(10⁸ CFU) was achieved with the effect of pelB and OmpF signal peptides.

1. A prokaryotic delivery carrier targeting a tumor, which is a chassis bacterium targeting the anaerobic area of a solid tumor, and comprises a polynucleotide for expressing a cytokine, wherein,
the cytokine is selected from the group consisting of interleukin 6, interleukin 10, interleukin8, interleukin 18, interleukin 33 and TNF-α, and
the polynucleotide is DNA or mRNA.

2. The prokaryotic delivery carrier of claim 1, wherein the chassis bacterium is selected from the group consisting of attenuated Salmonella typhimurium, attenuated Salmonella typhi, Escherichia coli and lactic acid bacteria.

3. The prokaryotic delivery carrier of claim 1, wherein the polynucleotide is DNA, and the carrier further comprises signal peptides pelB and OmpF fused to the DNA expressing the cytokine.

4. The prokaryotic delivery carrier of claim 1, wherein the polynucleotide is mRNA, and the mRNA further comprises an m7G capping structure at the 5′ end and poly A tail structure at the 3′ end.

5. The prokaryotic delivery carrier of claim 1, wherein when the polynucleotide is DNA, the prokaryotic delivery carrier comprises a promoter which is sensitive to tumor environment and other promoters which are constitutive promoter and inducible promoter.

6. The prokaryotic delivery carrier of claim 1, wherein the tumor is a solid tumor found in one or more of the following sites: cervix uteri, breast, prostate, intestinal tract, bladder, bone, skin, head or neck, liver, gall bladder, lung, pancreas, salivary gland, adrenal gland, thyroid, and cerebral ganglion.

7. The prokaryotic delivery carrier of claim 1, wherein the carrier is administered by intramuscular injection, intravenous injection, subcutaneous injection, intraperitoneal injection, intracerebral administration, intraoral administration, intranasal administration or oral administration.

8. A method for treating a tumor in a subject comprising administering the prokaryotic delivery carrier of claim 1 in combination with a therapeutic agent selected from the group consisting of chemical medicaments, chemotherapeutics, and immuno/targeting medicaments.

9. The method of claim 8, wherein the chemical medicaments are alkylating agents, alkane sulfonate, Az-cycloalkene medicaments, ethylamine and methylamine, nitrogen mustards, antibiotics, purine analogues, pyrimidine analogues, androsterones, antiadrenergics, topoisomerase inhibitors, capecitabine; the chemotherapeutics are anti-hormone agents regulating or inhibiting the effect of hormones on tumor; and the immuno/targeting medicaments are Gefitinib, Iressa, Lorlatinib, Erlotinib, Bevacizumab.

10. The method of claim 9, wherein the alkylating agent is thio-tepa and cyclophosphamide.

11. The prokaryotic delivery carrier of claim 5, wherein the inducible promoter is an IPTG inducible promoter or an aTc inducible promoter.

12. The prokaryotic delivery carrier of claim 6, wherein the tumor is colon cancer, breast cancer, melanoma, lung cancer, or glioblastoma.
